(19) United States
(12) Patent Application Publication
(10) Pub. No.: US 2024/0307600 A1
(43) Pub. Date: Sep. 19, 2024

(54) DIALYSIS TRACKING SYSTEM

(71) Applicant: Health Data Works, Inc., Jacksonville, FL (US)

(72) Inventors: Shannon L. Beardsley, Jacksonville, FL (US); Drew O'Keefe, Jacksonville, FL (US)

(73) Assignee: Health Data Works, Inc., Jacksonville, FL (US)

(21) Appl. No.: 18/576,966

(22) PCT Filed: Jul. 6, 2022
(86) PCT No.: PCT/US2022/073457

§ 371 Date: Jan. 5, 2024

Related U.S. Application Data
(60) Provisional application No. 63/203,039, filed on Jul. 6, 2021.

(51) Int. Cl. A61M 1/36; G16H 10/60; G16H 30/20

(52) U.S. Cl. CPC A61M 1/3656; G16H 10/60; G16H 30/20; A61M 2205/502

(57) ABSTRACT

Various embodiments disclosed relate to methods for producing an overlay of previous dialysis administration locations and access point events. The present disclosure includes a method of graphically producing the overlay with markers indicating one or more dialysis access points, where each of the markers conveys a location and a time of access. The overlay can include an image of the dialysis access location on which the access points are located.

### CROSS-REFERENCE TO RELATED APPLICATIONS

This application claims the benefit of U.S. Provisional Patent Application Ser. No. 63/203,039, filed on Jul. 6, 2021, the benefit of priority of which is claimed hereby, and which is incorporated by reference herein in its entirety.

### BACKGROUND

Dialysis is a process used in patients with kidney failure or malfunction to aid in removal of toxins, excess fluids, and waste products. In hemodialysis, a dialysis machine including a dialyzer membrane is used to filter toxins from the blood out through help of a dialysate and various sorbent materials. In hemodialysis, the patient's blood is cycled outside the body in an unfiltered state, and then cycled back in a chemically balanced state. In peritoneal dialysis, the natural lining of the peritoneum or abdomen is used in a similar process, with the air of a peritoneal catheter. Dialysis of both types helps remove unwanted substances from the bloodstream.

Patients who require dialysis are often regularly receiving dialysis for an extended period of time. Such patients often return to dialysis clinics repeatedly on a prescribed schedule for hemodialysis with a dialysis machine. Dialysis with a dialysis machine requires the patient's circulatory system so that blood may be removed from the body, cycled through the dialysis machine, and then returned to the body. Access points may be by way of catheters inserted into the patient's veins or may be by way of needles inserted into the patient's veins or a synthetic graft placed between an artery and a vein. Catheters, access veins and grafts may need to be changed from time to time for various reasons. If a vein or graft is to be accessed with a needle for dialysis, such access points are generally rotated to prevent complications.

However, the patient may choose to attend more than one dialysis clinic, change locations, change providers, or may undergo medical procedures related to the dialysis access at one or more facilities apart from the dialysis clinic. In this case, the dialysis technicians or nurses at the dialysis clinic or any location of medical treatment do not always have readily accessible dialysis records and may not be aware of prior access locations or interventions. Further, the specific site of needle placement may not be readily determined from one session of dialysis to the next either because adequately detailed records are not kept, or the records are not readily available for review by the individual obtaining access for dialysis.

### SUMMARY OF THE DISCLOSURE

Dialysis providers often see patients who are continuously or regularly receiving hemodialysis treatment, often multiple times per week. Dialysis providers try to avoid using the same dialysis access point over and over to prevent complications (e.g., scarring, clotting, collapsed veins) at the access point. However, information regarding such dialysis access points is not readily available to dialysis technicians at the point of care. For this reason, disclosed herein are aspects of methods and systems for recording, tracking, maintaining, communicating, and displaying information regarding locations and timing of dialysis vascular access points in patient records, including for technician use for regular hemodialysis treatment. This and similar dialysis information can be graphically depicted, for example, by an overlay on an arm (or representations of dialysis access relative to an anatomical area), with the overlay showing multiple markers of present and past dialysis access points. This overlay can be displayed to and used by the dialysis technician to aid in choosing an appropriate dialysis access point.

Disclosed is a method of data collection and software application configurations used in connection with dialysis treatment. For a particular patient, data can be inputted to a graphical user interface (GUI), such as a GUI provided by an interactive software application, for reference and access by multiple dialysis providers and medical personnel. The data can be represented within a graphical overlay or visualization of the patient's arm or other dialysis access area. The representation of the data can indicate for example a graft or other access portion, in addition to specific mapped access points of dialysis on the patient's anatomy. The data can be tracked at each visit, and access points can be time marked so that a technician can observe the application overlay which access points (e.g., recently used access points) should be avoided. These and related aspects of data tracking, management, visualization, and observation relevant to dialysis activities and related medical interventions are also disclosed.

In Example 1, a computer-implemented method of producing an overlay of dialysis access locations can include obtaining data indicating at least one dialysis access point of a patient, each of the at least one dialysis access point having an associated location on the patient and an associated access time, obtaining or producing an image of an anatomical area of the patient that is used for dialysis access, graphically representing the at least one dialysis access point as a marker on the image of the anatomical area at each corresponding dialysis access point, wherein the marker conveys both location and time of the dialysis access point, and displaying on a user interface, the overlay to a user, including the marker, on the image of the anatomical area.

Example 2 can include Example 1, wherein obtaining the image comprises obtaining a photographic image of the anatomical area of the patient, the photographic image captured with a camera.

Example 3 can include any of Examples 1-2, wherein obtaining the image comprises producing a computer-generated image of the anatomical area of the patient.

Example 4 can include any of Examples 1-3, wherein the marker corresponds to a keep-out area within the anatomical area to avoid for new administration of dialysis.

Example 5 can include any of Examples 1-4, wherein one or more of size, color, or shape of the marker are based on characteristics of the keep-out area.

Example 6 can include any of Examples 1-5, wherein the marker comprises a symbol associated with the access time of each corresponding dialysis access point.

Example 7 can include any of Examples 1-6, further comprising graphically representing a second dialysis access point with a second marker, wherein the second marker comprises a second symbol associated with the date of dialysis administration at the second dialysis access point.

Example 8 can include any of Examples 1-7, wherein the marker comprises a color associated with the date of each corresponding dialysis access point.

Example 9 can include any of Examples 1-8, further comprising graphically representing a second dialysis access point with a second marker, wherein the second marker comprises a second color associated with the access time at the second dialysis access point.

Example 10 can include any of Examples 1-9, wherein the marker is positioned based on location coordinates of the access point within coordinates of the image of the anatomical area.

Example 11 can include any of Examples 1-10, further comprising superimposing a radiographic layer on the image, the radiographic layer comprising another anatomical depiction of the anatomical area.

Example 12 can include any of Examples 1-11, further comprising superimposing a text overlay on the image, the text overlay comprising annotations or notes associated with the at least one dialysis access point.

Example 13 can include any of Examples 1-11, further comprising graphically representing one or more medical incidents on the image, wherein the one or more medical incidents are associated with a graft, stent, or other medical event of interest.

Example 14 can include any of Examples 1-13, further comprising recording a new dialysis access point after a new administration of dialysis.

Example 15 can include any of Examples 1-14, further comprising updating the overlay by graphically representing the new dialysis access point as a new marker on the image of the anatomical area at the corresponding new dialysis access point, wherein the new marker conveys both location and date of the new dialysis access point.

Example 16 can include any of Examples 1-15, further comprising adding information to the overlay to indicate which dialysis access points were entered at which times.

Example 17 can include any of Examples 1-16, wherein the anatomical area comprises an arm, a leg, a limb, or another location on a patient's body for administration of dialysis.

Example 18 can include any of Examples 1-17, wherein obtaining data comprises receiving the data indicating the at least one dialysis access point from a database.

Example 19 can include any of Examples 1-18, wherein the database stores data corresponding to performance metrics for prior access by each clinician at the at least one dialysis access point, the performance metrics being determined based on avoidance of prior dialysis access points by each clinician.

Example 20 can include any of Examples 1-19, wherein the method is performed by a server computing system, and wherein displaying the overlay to the user comprises providing the image of the anatomical area from the server computing system to a client computing system operated by the user.

Example 21 can include any of Examples 1-20, wherein the method is performed by a client computing system, and wherein displaying the overlay to the user comprises providing the image of the anatomical area in a graphical user interface provided by the client computing system.

Example 22 can include any of Examples 1-21, wherein the graphical user interface comprises a website interface.

Example 23 can include any of Examples 1-22, wherein the graphical user interface comprises a software application, and wherein the client computing system is a mobile computing device.

Example 24 can include any of Examples 1-23, wherein the method is performed by an augmented reality device, wherein obtaining the image of the anatomical area comprises capturing the image of the anatomical area from a real-world view of the anatomical area, and wherein displaying the overlay to the user comprises augmenting the real-world view of the anatomical area in a display provided by the augmented reality device.

Example 25 can include any of Examples 1-24, wherein the augmented reality device is a headset, smart glasses, or a smartphone with integrated camera and display.

Example 26 can include a non-transitory machine-readable medium including instructions, which when executed by a processor of a computing system, cause the computing system to: obtain data indicating at least one dialysis access point of a patient, each of the at least one dialysis access point having an associated location on the patient and an associated time: obtain or produce an image of an anatomical area of the patient that is used for dialysis access: graphically represent the at least one dialysis access point as a marker on the image of the anatomical area at each corresponding access point, wherein the marker conveys both location and time of the dialysis access point; and display a graphical overlay to a user, including the marker, on the image of the anatomical area.

Example 27 can include Example 26, wherein the image is a photographic image of the anatomical area of the patient captured from a camera or a computer-generated image of the anatomical area of the patient.

Example 28 can include any of Examples 26-27, wherein the marker comprises a symbol indicating the access time of each corresponding dialysis access point.

Example 29 can include any of Examples 26-28, wherein the marker comprises a color indicating the access time of each corresponding dialysis access point.

Example 30 can include any of Examples 26-29, wherein the anatomical area comprises an arm, a leg, a limb, or another location on a patient's body for administration of dialysis.

Example 31 can include any of Examples 26-30, wherein the marker is positioned based on location coordinates of the access point within coordinates of the image of the anatomical area.

Example 32 can include any of Examples 26-31, wherein the instructions, when executed, further cause the processor to graphically represent one or more medical incidents on the image, wherein the one or more medical incidents comprise a graft, stent, or other medical location of interest.

Example 33 can include any of Examples 26-32, wherein the instructions, when executed, further cause the processor to record a new dialysis access point after a new administration of dialysis and update the overlay accordingly.

Example 34 can include any of Examples 26-33, wherein the computing system is a server computing system, and wherein the operations to display the overlay to the user comprises operations to provide the image of the anatomical area from the server computing system to a client computing system operated by the user.

Example 35 can include any of Examples 26-34, wherein the computing system is a client computing system, wherein the operations to display the overlay to the user comprises operations to provide the image of the anatomical in a graphical user interface output by the client computing system, wherein graphical user interface comprises a website interface or a software application.

Example 36 can include a cloud computing system, comprising: a communications device configured to perform communications with a client computing system using at least one application programming interface; a memory device configured to store instructions; and processing circuitry configured to execute the instructions, wherein the instructions, when executed, cause the cloud computing system to host and operate at least one database and the at least one application programming interface, the at one application programming interface to communicate data from the database to the client computing system, to enable use of a graphical user interface at the client computing system for producing an overlay of dialysis access locations according to any of the methods of Examples 1 to 25.

Example 37 can include a server computing system, comprising: a memory device configured to store instructions; and processing circuitry configured to execute the instructions, wherein the instructions, when executed, cause the server computing system to generate and communicate data from the server computing system to a client computing system, to enable use of a graphical user interface at the client computing system for producing an overlay of dialysis access locations according to any of the methods of Examples 1 to 25.

Example 38 can include a client computing device, comprising: a memory device configured to store instructions; and processing circuitry configured to execute the instructions, wherein the instructions, when executed, cause the client computing device to access and communicate data with at least one application programming interface at a server computing system, the data to enable use of a graphical user interface at the client computing device for producing an overlay of dialysis access locations according to any of the methods of Examples 1 to 25.

Example 39 can include a client computing device, comprising: a memory device configured to store instructions; and processing circuitry configured to execute the instructions, wherein the instructions, when executed, cause the client computing device to implement a software application, the software application to provide a graphical user interface at the client computing device for producing an overlay of dialysis access locations according to any of the methods of Examples 1 to 25.

Example 40 can include an augmented reality device, comprising: an imaging device: a display device: a memory device configured to store executable instructions for an augmented reality (AR) software application; and processing circuitry configured to execute the instructions, wherein the instructions, when executed, cause the augmented reality device to implement the AR software application, the augmented reality software application to provide an AR graphical user interface on the display device, for producing an overlay of dialysis access locations with AR according to any of the methods of examples 1 to 25.

Example 41 can include a method of administering dialysis, the method comprising: collecting dialysis data for a patient, including one or more locations of dialysis treatment on the patient. wherein each of the one or more locations of dialysis treatments has an associated time record indicating how old the location is: producing an overlay for view on a user interface, the overlay indicating the one or more locations of dialysis treatment on the patient and the associated time record; determining, based on the overlay, an appropriate treatment location for dialysis treatment separate from one or more locations having a recent time record; and administering dialysis at the treatment location on the patient.

Examples 42 can include Example 41, wherein collecting the dialysis data further comprises collecting information about a location of a graft or other access point, wherein the overlay further indicates the location of the graft or other access point.

Example 43 can include any of Examples 41-42, further comprising updating the overlay after administering dialysis by adding data about the treatment location to the patient with an associated time record.

Example 44 can include any of Examples 41-43, wherein the treatment location comprises a new location.

Example 45 can include any of Examples 1-44, wherein the treatment location comprises one of the one or more locations having a time record that is older relative to the other of the one or more locations.

Example 46 can include any of Examples 41-45, further comprising color coding the one or more locations correlated to their time records, wherein the color coding indicates to a technician which of the one or more locations are preferable for administering dialysis.

Example 47 can include any of Examples 41-46, wherein producing the overlay comprises producing a map of the patient's arm including the one or more locations of dialysis treatment.

Example 48 can include a non-transitory machine-readable medium including instructions, which when executed by a processor of a computing system, cause the computing system to: obtain dialysis data for a patient, including one or more locations of dialysis treatment on the patient, wherein each of the one or more locations of dialysis treatments has an associated time record indicating how old the location is; generate an overlay for view on a graphical user interface, the overlay indicating the one or more locations of dialysis treatment on the patient and the associated time record: output information to enable a determination of an appropriate treatment location for dialysis treatment separate from one or more locations having a recent time record; and output information to enable an administration of dialysis at the treatment location on the patient.

Example 49 can include Example 48, wherein collecting data further comprises collecting information about a location of a graft or other access point, wherein the overlay further indicates the location of the graft or other access point.

Example 50 can include any of Examples 48-49, the instructions further to cause the computing system to update the overlay after administering dialysis by adding data about the treatment location to the patient with an associated time record.

Example 51 can include any of Examples 48-50, wherein the treatment location comprises a new location.

Example 52 can include any of Examples 48-51, wherein the treatment location comprises one of the one or more locations having a time record that is older relative to the other of the one or more locations.

Example 53 can include any of Examples 48-52, the instructions further to cause the computing system to perform coding of the one or more locations correlated to their time record, wherein the coding indicates to a technician which of the one or more locations are preferable for administering dialysis.

Example 54 can include any of Examples 48-53, wherein generating the overlay comprises producing a map of the patient's arm including the one or more locations of dialysis treatment.

Example 55 can include a cloud computing system, comprising: a communications device configured to perform communications with a client computing system using at least one application programming interface: a memory device configured to store instructions; and processing circuitry configured to execute the instructions, wherein the instructions, when executed, cause the cloud computing system to host and operate at least one database and the at least one application programming interface, the at one application programming interface to communicate data from the database to the client computing system, to enable use of a software application at the client computing system for supporting the methods of administering dialysis according to any of the methods of Examples 41 to 47.

Example 56 can include a server computing system, comprising: a memory device configured to store instructions; and processing circuitry configured to execute the instructions, wherein the instructions, when executed, cause the server computing system to generate and communicate data from the server computing system to a client computing system, to enable use of a software application at the client computing system for supporting the methods of administering dialysis according to any of the methods of Examples 41 to 47.

Example 57 can include a client computing device, comprising: a memory device configured to store instructions; and processing circuitry configured to execute the instructions, wherein the instructions, when executed, cause the client computing device to implement a software application, the software application adapted to support the methods of administering dialysis according to any of the methods of Examples 41 to 47.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a block diagram of a dialysis data collection and information presentation system according to an example.

FIG. 2 illustrates a diagram of various dialysis data processing sources according to an example.

FIGS. 3A-3B illustrate images of a patient arm and generated overlays with dialysis data according to an example.

FIG. 4 illustrates a dashboard associated with an overlay of dialysis data according to an example.

FIG. 5 illustrates a flow chart of a method of administering dialysis in an example.

FIG. 6 illustrates a block diagram of a server and client computing system for implementing the computing and processing techniques discussed herein, according to an example.

FIG. 7 illustrates a block diagram of an example computing machine upon which any one or more of the techniques or methodologies discussed herein may be implemented.

### DETAILED DESCRIPTION

The present disclosure describes, among other things, a method and system for recording and using a dialysis visualization to determine the location and age of various dialysis access points on a patient. Various aspects of dialysis information tracking, and the representation and use of medical data related to dialysis procedures and medical interventions, are also disclosed.

In dialysis care, tracking previous dialysis points of access and care can be challenging. When a patient has kidney failure and begins regular dialysis treatment, the patient can be fitted with a catheter for access to the circulatory system, or with a fistula or graft for dialysis needle access to the circulatory system. Generally, fistulas or grafts have a limited lifetime, and can fail due to recurrent stenosis, scarring, thrombosis or other flow challenges. As a graft or fistula fails, other areas of access are used on the patient's body. Preferably, access is maintained for a prolonged period of time using any particular access graft or arteriovenous fistula, but problems such as recurrent stenosis, scarring, thrombosis or other flow challenges can force creation of additional new access locations. Rotation of needle access points within a particular access graft or arteriovenous fistula site can reduce complications within the access such as recurrent stenosis, scarring, thrombosis or aneurysmal dilation which may otherwise tend to occur due to repeated localized trauma from repeated needle punctures in or near the same location.

Tracking these access points is often incomplete or haphazard. The patient may receive dialysis care and oversight from a variety of providers, technicians, or clinics. A given technician administering dialysis may not be aware or have access to data indicating prior access points and complications from such access points. For example, the technician may not be aware that the patient has had a stent inserted at a particular location, a balloon dilation of a stenosis conducted at another location, or a stent placed at another location. Granular information regarding a patient's dialysis treatment history is often hard to come by. The technician also may not be aware of medical interventions performed on the patient's dialysis access graft or arteriovenous fistula while at medical facility other than the dialysis clinic, procedures which could include a de-clot of the graft or arteriovenous fistula, thrombolysis of the graft or arteriovenous fistula, balloon angioplasty (dilation) of a stenosis or placement of a stent.

The methods and systems discussed herein allow for tracking of such dialysis treatment information for a more holistic view over the treatment lifetime of a dialysis patient. For example, the methods and systems discussed herein can track information related to how long a patient has had a catheter, the most recently used access points, history of infections at respective access points, what kind of fistulas or grafts have been inserted and used, and potential next access points with beneficial flow. The methods and systems can also enable tracking of normal or adverse events at the patient's access points, such as punctures or the generation of grafts, clotting (and de-clotting), and other events or structures. Tracking access points and providing a visualization or illustration of this information and events can allow for the technician to make an educated decision on where to next attempt an access point for dialysis treatment.

As discussed herein, “anatomical area” can include a portion of a patient's body being used or accessed for administration of dialysis. For example, an anatomical area can include an arm or leg used for dialysis access. In some cases, an anatomical area can be a portion or designated section of such patient anatomy.

As discussed herein, “access point” can include a location or site on a patient's body which the technician or doctor (or other caregiver/medical assistant) uses to administer dialysis. The site can be a place of puncture or incision to connect a dialyzer or other dialysis instrumentation to a patient's circulatory system. An access point can be actually used or attempted for use for dialysis administration.

As discussed herein, “overlay” can include an image, layer, or graphic, that represent coordinates on the human body, such as at an anatomical area. Various points of interest, such as markers representing access points or other medical items. In some cases, an overlay can be layered on top of or with other images. In some cases, an overlay can be integrated with other images.

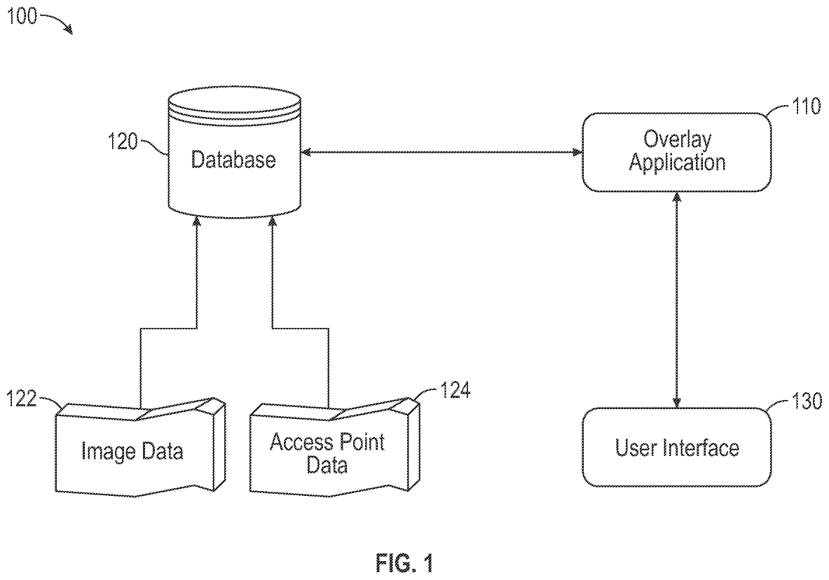

FIG. 1 illustrates a diagram of a dialysis data collection and information presentation system in an example. The system 100 can include an overlay application 110 (e.g., a software application), database 120 (e.g., provided by a medical record system), sources of dialysis data 122, 124, (e.g., image data 122 providing photographs or representations of respective patient anatomical areas, access point data 124 providing metadata and coordinates of access points relative to the patient anatomical areas) and user interface 130 (e.g., a graphical user interface generated or provided from the overlay application 110). The system 100 can be accessed and used, for example, by a dialysis provider or clinician (e.g., physician or nurse), such as for administration of dialysis or for medical care associated with dialysis.

The overlay application 110 can be used to produce a graphical display of dialysis treatment history for use by a dialysis technician or clinician, such as in planning, review, or execution of patient dialysis treatment. The overlay application 110 can communicate with the database 120 to receive data and produce an overlay image for view and interaction on the user interface 130. The overlay application 110 can be used to produce an overlay image using the various data points (e.g., coordinates or locations of access points) received from the database 120, and graphically present that data regarding prior dialysis treatment to a user within the user interface 130. In an example, the overlay application 110 is a cloud-, web-, or server-hosted application or service, which communicates with a client-based instance of the user interface 130. In another example, the overlay application 110 is a client-based software application that executes on a client computing device and communicates with a local or remote instance of the database 120 and the data sources 122, 124. It will be understood that a variety of implementations for client, server, and synchronized and distributed services and data storage may be employed.

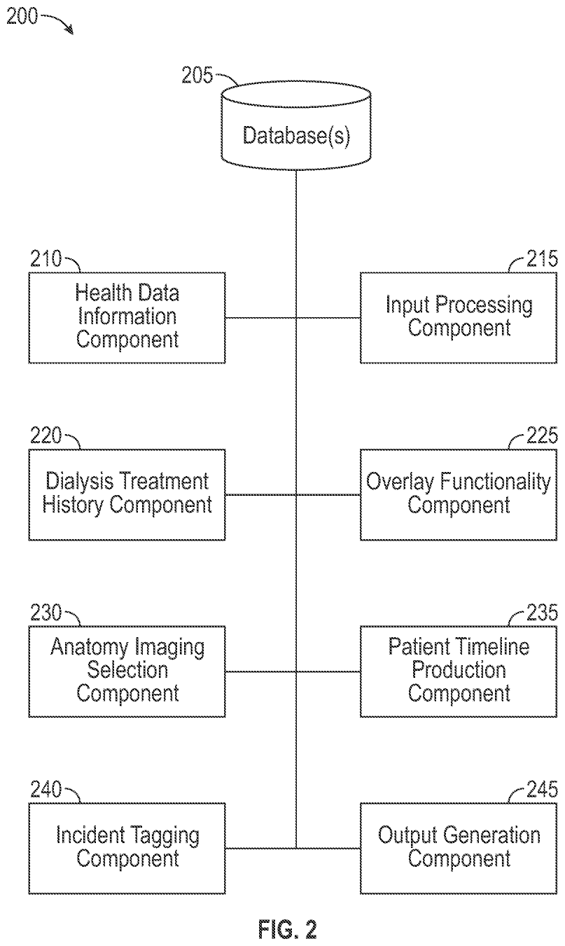

The database 120 can include a library of information, such as related to a specific patient and the patient's dialysis treatment and maintain such a library for multiple patients or subjects associated with a dialysis treatment site. The database 120 can be hosted via a cloud service, or a local service (including, a local instance of a database which synchronizes the library with a remote source). The database 120 can be dynamically updated with new data, including in real-time during the performance of a dialysis procedure. The database 120 can allow for access to patient dialysis data from a variety of medical providers, clinics, dialysis providers, and related entities. The data sources 122, 124, incoming to the database 120 can include, for example, patient dialysis treatment history, patient images, clinician or technician notes, or other data, such as discussed in more detail below with reference to FIG. 2.

The database 120 can be in communication with the overlay application 110 to provide relevant data so the overlay application 110 can produce one or more images or overlays for use by the dialysis provider or clinician. Other data sources (not shown) may include electronic medical record (EMR) or electronic health record (EHR) systems and related interfaces, such as those maintained by a medical provider, insurance company, government health authority, dialysis service provider, etc. In a further example, the database 120 also stores data which can be used to compile, track, or determine performance metrics for prior access by each clinician during dialysis procedures. For instance, such performance metrics may be calculated based on avoidance of prior dialysis access points by each clinician, or other safety or usage protocols established for a patient, clinician, or medical practice.

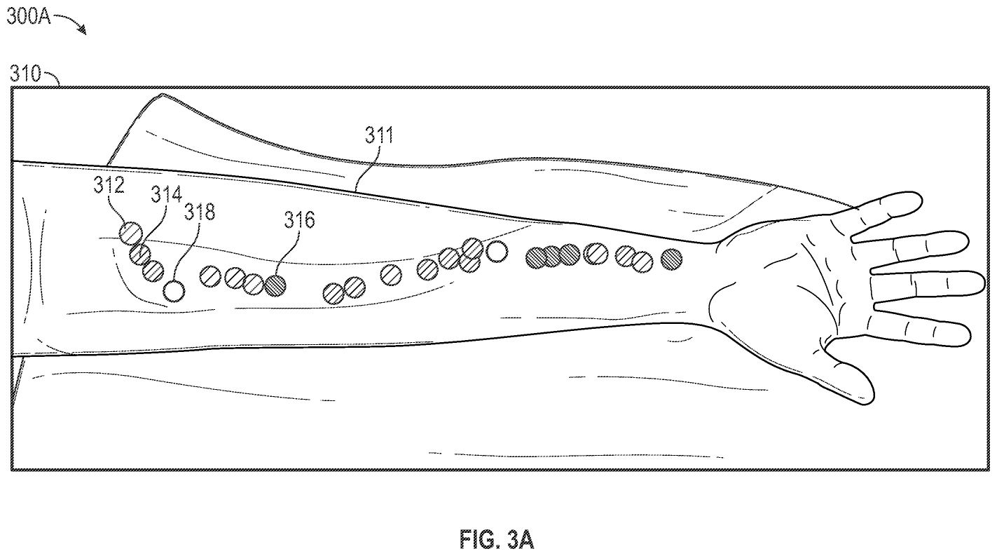

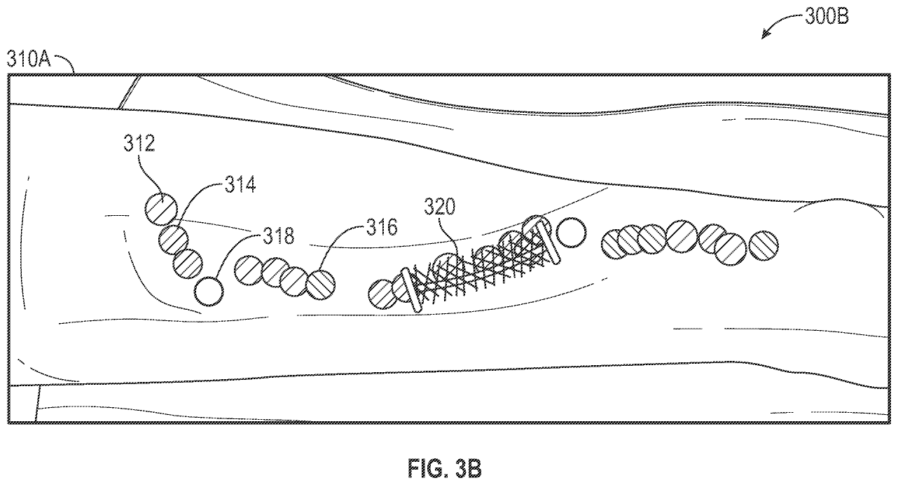

The user interface 130 can be used to display images and the produced overlay for a user. Example produced overlays are shown and discussed in more detail with reference to FIGS. 3A-3B below. The user interface 130 can also be used to receive and display new information related to a current or historical dialysis procedure, such as from the marking of a new access point occurring at the time of dialysis treatment. The user interface 130 can also be used to receive and display information related to a current or historical medical intervention, condition, or event, such as the insertion of a stent or graft, clotting or declotting event, or the like.

FIG. 2 illustrates a block diagram of an example of a system 200 for dialysis information management and tracking. The system can include a variety of data processing components, such as a patient information data record 205, health data information component 210, input processing component 215, dialysis treatment history component 220, overlay functionality component 225, anatomy imaging selection component 230, patient timeline production component 235, incident tagging component 240, and output generation component 245.

Each of the data processing components within the system 200 may be provided by different interfaces (e.g., application programming interfaces (APIs)), software modules or units, or otherwise executed and operated by a software system implementation. The data processing components operated within the system 200 in FIG. 2 can be used, for example, to receive, extract, display, and store data related to patient dialysis treatment, to implement the graphical inputs, outputs, and data processing aspects related to the dialysis use cases discussed herein. For example, during a patient's lifetime treatment with dialysis, data can be processed, recorded, and stored by the system 200 for later reference and use. The system 200 can provide functionality that is beneficial for both dialysis providers and clinician, as it can allow for real time and historical tracking of patient health and treatment data.

The patient information data record 205 can include a variety of data that may be used by the system 200 while in operation. For example, the patient information data record 205 may include patient data including patient anatomy data, patient dialysis history data, patient dialysis access points, patient timeline, past patient incidents or events, and the like. Such data may be provided in a format that is custom to the system 200, or in a format that is more generally applicable to a medical data record or medical data logging system.

The health data information component 210 can access or provide a repository of healthcare information in connection with the system 200. For example, the health data information component 210 can be accessed through the system 200 and verify compliance or security measures defined according to the Health Insurance Portability and Accountability Act (HIPPA), such as to allow a dialysis provider to access protected health information (PHI) within patient health records. Patient health records accessible within the health data information component 210 can include, for example, a holistic view or timeline of relevant or background medical information for the patient, such as previous diagnoses, medications, visits, and related medical conditions or incidents. The system 200 can convey updated dialysis information to the health data information component 210 and allow access to such updated information by other clinics or providers.

The input processing component 215 can receive and process incoming data related to use of the system 200. For example, the input processing component 215 can receive a query from a clinician via the user interface. For example, the clinician may desire to search or review specific data regarding the patient's dialysis history, such as access points, timing, or incidents. The input processing component 215 can intake the query, such as in a text format, and prepare that query, such as through formatting or reordering, to other components of the system 200 to access, retrieve, and produce the data, images, or other commentary desired by the clinician. The input processing component 215 can also route corresponding commands to other components of the system 200.

The dialysis treatment history component 220 can collect, organize, and extract patient dialysis treatment information and data. The dialysis treatment history component 220 can, for example, include functionality to collect and store information such as timing of dialysis treatments, access points for those treatments, where the treatment was given, at what clinic with which technician, the length of time of that treatment, and any incidents that occurred. In some cases, the dialysis treatment history component 220 can further store and produce information related to a specific dialysis treatment session such as flow rate of blood, the type of access point used (such as a graft or fistula), and techniques used by the technician (such as balloons, clearing of clogging, etc.), or other information. In some cases, the dialysis treatment history component 220 can further calculate or provide ratings, such as flow ratings, access ratings, or other grading of dialysis treatments for clinician use. The dialysis treatment history component 220 can be in communication with the overlay functionality component 225 to produce a graphical overlay, as discussed in the following paragraphs.

The overlay functionality component 225 can process data inputs from the input processing component 215, the dialysis treatment history component 220, and other components of the system 200 to produce a graphical overlay such as those shown and discussed below with reference to FIGS. 3A-3B. The overlay functionality component 225 can, for example, obtain information about recent dialysis treatment access points, including where the access points are located on a patient's anatomical area, such as an arm, and plot the access points on a representation of the anatomical area according to coordinates (e.g., points or ranges) within some spatial coordinate space. The overlay functionality component 225 can further obtain information about the timing of access point use and use such information to graphically represent the recency of each access point, such as with colors, symbols, or tags within the overlay. In some cases, the overlay functionality component 225 can extract images of the patient anatomy from the anatomy imaging selection component 230, and place the overlay over the image, such as of a patient arm.

The anatomy imaging selection component 230 can collect, access, and distribute images of one or more patient anatomical areas (e.g., a patient arm) that may be used or tracked for dialysis access. Anatomy imaging data can include, for example, patient radiographic images (e.g., X-ray, CT, MRI scans), medical visualization images, and the like, in 2D or 3D, which represent aspects of one or more of the skeletal systems, vascular (circulatory) system, and other anatomical components at or near the points of access used for dialysis treatment. In some cases, the anatomy data can include layers of images, such as to allow the user to retrieve from the patient information data record 205 various images of anatomical areas for presentation on a user interface. In an example, a user could desire to see the layout of the circulatory system in the patient and retrieve from the patient information data record 205 anatomy data in the form of a radiographic image displaying that system onto the user interface, such that the images align with the produced overlay. Similarly, the user can retrieve one or more bone images, and display them in concert with the circulatory system, or in the alternative. In some cases, the anatomy data can include other types of imaging, such as computer-generated images, outlines, visualizations, or models of the patient's anatomy.

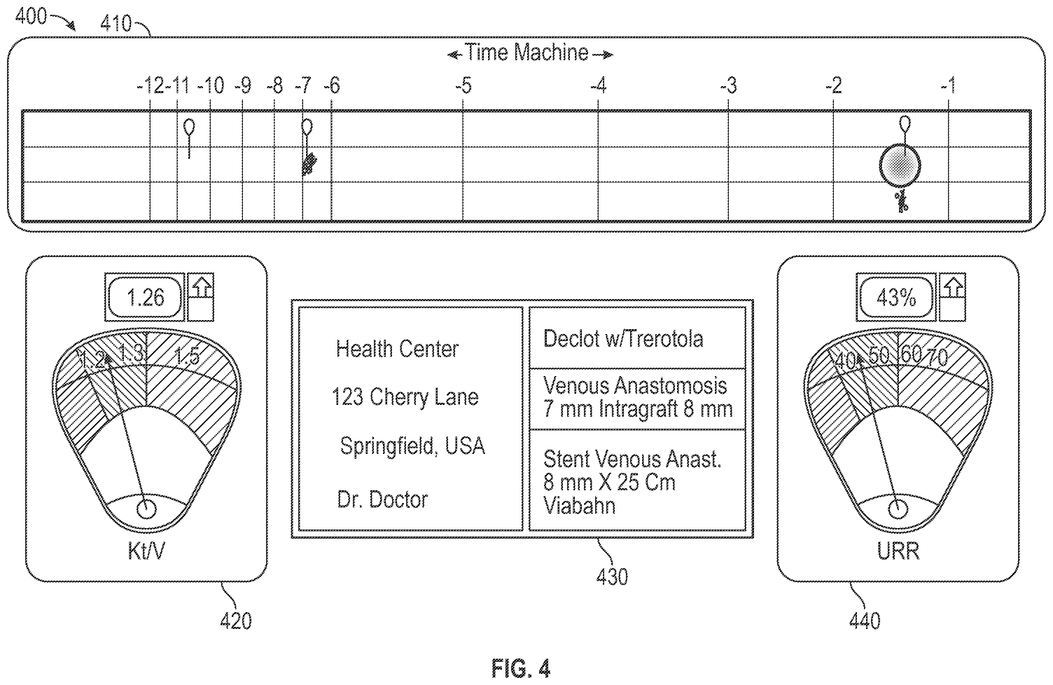

The patient timeline production component 235 can receive data from other components and produce a patient dialysis dashboard analysis, such as the example timeline shown and discussed with reference to FIG. 4 below. The patient timeline production component 235 can intake data, such as related to dialysis access points, flow rate at those access points, incidents that have occurred during past dialysis treatments, and other information. The patient timeline production component 235 can synthesize this information and production one or more graphical representations of the data, such as tables, charts, dials, or other graphics to present on the user interface. In some cases, the patient timeline can include one or more user interactive graphical representations.

The incident tagging component 240 can produce text describing or labeling various dialysis or medical incidents graphically represented on an overlay or dashboard. The incident tagging component 240 can communicate with other components of the system 200, such as the dialysis treatment history component 220, the overlay functionality component 225, and the patient timeline production component 235, to identify and collect information regarding medical incidents. For example, medical incidents can include use of a balloon during dialysis treatment, placement of a stent, or other medically significant events related to dialysis treatment or care. The incident tagging component 240 can organize information related to these incidents and present the information as desired to the user with the overlay, timeline, and/or dashboard. For example, a specific incident can be graphically represented on the dashboard as a marker on a timeline. The incident tagging component 240 can present specific information related to that incident as a pop-up window with details regarding the incident, such as when a user clicks on the incident graphically represented in the dashboard.

The output generation component 245 can format output or otherwise prepare the outputs generated by components of the system 200 for display on the user interface. The output generation component 245 may present the overlay produced by the overlay functionality component 225 using data from the dialysis treatment history component 220 layered with imaging from the anatomy imaging selection component 230 and include incident tags produced by the incident tagging component 240 on the overly. The output generation component 245 may additionally or alternatively output a dashboard produced with the patient timeline production component 235 using data from the dialysis treatment history component 220. The outputted dashboard or timeline can include tagged incidents from the incident tagging component 240.

In system 200, data can be received into the patient information data record 205 from health data information component 210 and the input processing component 215. Inputs from a user can include text and data related to dialysis treatment. Data can be extracted from the patient information data record 205 by the components 220, 225, 230, 235, 240, for presentation in one or more graphical representations via output generation component 245.

It will be understood that data can be displayed from the system 200 in a variety of formats or interactive representations, including overlays, images, customizable dashboards, such as including charts and dials, and other graphical representations and controls for the output of dialysis treatment data. The use of system 200 can allow for patients, physicians, and dialysis clinics to see a holistic view of the patient's dialysis treatment history, and observe patterns or trends, such as flow rates and patterns of treatment. In some cases, this can allow the caregiver to see areas of potential challenges and intervene ahead of time before failure occurs. In some cases, this can allow for dialysis treatment centers to also observe different levels of performance of their technicians, review whether grafts of individual patients are properly functioning, and observe treatment results or complications performed at other locations.

FIGS. 3A-3B illustrate examples of a patient arm image with an overlay for representing dialysis data. The example image can be produced, for example, by the system 200, and presented on a graphical user interface for review and interaction by a dialysis provider or associated medical personnel. The example overlay 300A or 300B is depicted as including an image 310 of an arm 311 with indicators of dialysis access points 312, 314, 316, 318, and graft 320. The overlay 300A can additionally or alternatively include other dialysis and treatment information, or anatomical information such as radiographic images, commentary from clinicians or previous technicians, and other information. Such information or anatomical or radiographical images can be layered, superimposed, or positioned on or under the overlay.

In the example overlay 300A of FIG. 3A, prior dialysis access point indicators 312, 314, 316, and 318 are shown on top of the image 310. The image 310 can be, for example, a photo of the patient's arm, or a computer-generated representation of such an anatomical area. The image 310 can, in an example, include a coordinate space, such as x-y or cartesian coordinates, which can be used to pinpoint various locations (e.g., areas or ranges) on the patient anatomy. The various coordinates can be used to track the access points such that a technician can mark, locate, and observe the corresponding locations on a patient's physical arm when conducting the dialysis procedure.

In the example overly 300, access point indicators 312, 314, 316, and 318 can represent access points on the patient's arm used for dialysis at various times. For example, access point indicator 312 can represent access points that were most recently used, such as in the last two weeks. The access point indicator 312 can be, for example, color-coded, or associated with a particular symbol, such that the indicator can be easily associated with a particular time frame (e.g., date range, period of time, etc.). Similarly, the access point indicator 314 can be, for example, color-coded (or stamped with a particular symbol) to indicate that the access point was used less recently, such as being accessed in a two week to one month ago time frame. Access point indicator 316 can be visually indicated to be associated with a different time frame, for example, such as being accessed more than one month ago. By comparison, the access point indicator 318 can be, for example, potential access points that have not yet been used, or have not been used within some time period.

The access point indicators 312, 314, 316, and 318 can be static, or can be automatically updated as time moves forward. For example, if access point indicator 312 was used within the last two weeks, such an indicator can be color-coded red. Once two weeks pass from the time of use, the access point indicator 312 can be switched to orange (e.g., used between two to four weeks ago). Similarly, once four weeks pass from the time of use, the access point indicator 312 can be switched to yellow (e.g., used between four to six weeks ago). After six weeks have passed, the access point indicator 312 can be switched to white. Thus, the time frame of access point use can be visually represented for the user, including for multiple access points and areas of access points. In some cases, different symbols, shadings, shapes, or sizing of the markers representing the access points can be used instead of (or, in addition to) color-coding. In some cases, the markers can be of varying sizes to indicate zones or coordinates.

The markers may be generated or presented to help clearly indicate one or more keep-out areas (keep-out “zones”) on the patient's anatomy to avoid for new administration of dialysis, such as time-based keep-out zones based on recency of access. The size, color, or shape of the marker may be determined from the characteristics of the keep-out area. For example, a displayed marker may specifically define a size or zone around the prior access point within which a new needle access should not occur within a certain time frame (represented by a combination of the presence of the marker, and color or attributes of the marker/symbol).

Often, two access points are used for one treatment, such that the first access point is arterial access and the second is venous access. In this case, these two access points may be coordinated together. The size or shape of the access point indicator may also be coordinated to allow a presentation of indicators in a quickly-to-understand manner.

The overlay 300A can be displayed for a user via a graphical user interface. When reviewing the overlay 300, the user, such as a dialysis technician or a clinician, can review the overlay to determine the most recent dialysis access points used on the patient, and potentially which dialysis access points would have the highest chance of success without clotting or other challenges.

In some examples, the overlay 300A can additionally depict stents, grafts, fistulas, or other components related to dialysis. For example, in FIG. 3B, a graft 320 is shown on the patient's arm. In this case, the graft can be graphically displayed through shapes or symbols indicating where (and, at what coordinates) the graft has been implanted. In some cases, the user can interact with the image, such as by touch or click, to display additional information about the graft. For example, pop-up information can include data about the function of the graft, such as current flow rates and documented sessions using the graft. In some cases, the information can indicate which portions of the graft have good flow or poor flow (with the amount of flow being assessed by the clinician or by some metric), or the information can indicate if and where a balloon has previously been used in the graft.

In some cases, the overlay 300A can additionally include radiographic information. In this case, radiology images (or visualizations based on radiology images) of the patient's anatomy, such as bones, circulatory system, muscles, etc., can be layered with the overlay 300A to provide the user with a more holistic view of the patient anatomical area. In an example, the doctor or technician can toggle between various layers to show, for example, the circulatory system or the skeleton, in conjunction with the dialysis access points. This can allow for specific tailoring of the overlay for the purpose of treatment and analysis. In this case, the overlay can be constructed using radiology data collected from imaging procedures of the patient. Such data can be updated periodically as desired. In another example, the doctor or technician can control varying translucency of overlaid layers to see information from various layers within the same overall image, thus allowing additional information (or information from different sources) to be displayed at the same time.

In some cases, the direction of blood flow and type of dialysis access can be indicated on the overlay. For example, an arterial and venous side can be indicated by colors or arrows in the blood flow. Similarly, types of access, such as grafts and fistulas can be indicated in the overall display with the placement and use of different symbols, colors, labels, and the like.

In some cases, the overlay 300A can be projected onto a static image of a patient's arm, such as a photograph, with dialysis access points being tracked according to an x-y coordinate of the photograph image. The coordinates can be, for example, a 1000×1000 2-D graph (e.g., 1000×1000 pixels). In some cases, the coordinate system can be a 3-D rendering. In some cases, the image of the patient's arm can be generated from a computer-generated model, or an enhanced image or simulation (e.g., generated or enhanced using a trained artificial intelligence (AI) model) based on scans, measurements, or other information about the patient's arm. In some cases, the overlay 300A can be configured for use over one or more layers, such as for toggling visibility or fading of structures from radiology images or computer-generated visualizations.

In some cases, the overlay 300A can be configured for use in an augmented reality (AR) setting in conjunction with a live camera (e.g., situated to image the patient's arm, such as provided from a stationary camera, a camera on an AR device such as glasses, AR-capable smartphone or tablet, etc.). As an example, a clinician or technician may wear an AR headset (e.g., glasses with an integrated camera) while looking at the patient's arm and use an AR application to dynamically display and adapt the overlay 300A into a series of AR objects presented in the field of vision of the patient's arm. As another example, a clinician or technician may hold an AR-enabled electronic device (e.g., smartphone or tablet) over a patient's arm to display real-time view from an opposite-facing camera on the device screen, as an AR application dynamically displays and adapts the overlay 300A into AR objects dynamically positioned within the camera image of the patient's arm.

An AR overlay may display symbols or indicators for access points in addition to superimposed text, images, or features relevant to the patient's medical history or medical procedures. Various real-world coordinates of the patient's anatomy may be observed or detected with the camera (or other imaging component) of the AR device, or such coordinates may be calculated or fit to the patient using AI models or AI-enhanced techniques for detecting and localizing the AR objects in a user's field of view. The superimposition of the overlay data with a real-time, real-world view of the patient's arm may provide a highly efficient way to communicate information and history to the clinician or technician.

As noted, FIG. 3B depicts a version of the overlay 300B that includes a stent. In this overlay, the operator placing the stent (e.g., interventional radiologist) would have the capability to enter the stent information into the system, such that later technicians or providers would be able to see the stent in the overlay. The provider inserting the stent could provide bibliographic information regarding the stent, and “beginning” and “end” points of the stent. A stent image could be created between these inserted points, with a curvature for the stent as desired.

The overlay 300B can additionally include one or more incident reports or information tabs. For example, if a stent is included in the overlay 300, a user may be able to click on the stent in the image to pop up a window with stent information such as the facility the stent was provided, the operator who placed the stent, the date the stent was inserted, the brand and size of the stent, and a descriptive location, such as “venous anastomosis”. This could allow future providers to have more knowledge of the stent when treating the patient. In some cases, additional medical locations of interest can be graphically represented on the overlay 300.

Additionally, information regarding the medical locations of interests can be included with the overlay 300. For example, a user may click or select the stent shown on the overlay 300, and a pop-up or other window can indicate information such as the individual who placed the stent, the size, make, and model of the stent, and the date the stent was placed.

FIG. 4 illustrates a dashboard 400 associated with the overlay in an example. The dashboard 400 can include user interactive controls such as dials 420 and 440, and output information provided from bibliographic information 430 and timeline 410. The dashboard 400 can be produced based on patient dialysis treatment history data, such as from a database or data record as discussed above. The dashboard 400 can be displayed, for example, on or near the overlay image for dialysis provider or clinician reference and use.

The first dial 420 can, for example, be a dial representing an average measure of blood flow over previous dialysis treatments, such as in Kt/V. The Kt/V measurement represents flow and can be measured at a predetermined interval over the patient's dialysis treatments. As is understood, Kt/V can be used to numerically represent adequacy of dialysis treatment, with K representing a dialyzer clearance of urea, t representing dialysis time, and V representing a volume of distribution of urea (e.g., according to the patient's total body water). For example, Kt/V can be measured every week, or once every three dialysis treatments, or other consistent intervals as desired. On the first dial 420, where the needle is in the “green” rightmost portion, the flow can be considered in an acceptable range. Where the needle is in the “red” leftmost portion, the flow can be considered poor. The clinician or practitioner can set thresholds as desired. The box above the color range dial can show the most recent measured image, which will be from some prior dialysis session. The small box to the right with the arrow can demonstrate a trend that may exist over some number of prior sessions.

The second dial 440 is similar in set up to the first dial, but can represent urea reduction ratio (URR), a measure of urea clearance. The URR measurement can help determine how effective dialysis is for the patient. Other measurements related to the effectiveness or outcome of a dialysis treatment may also be tracked.

The bibliographic information 430 can include information for quick reference, such as patient and doctor information, clinic information, and notes on most recent events, such as dialysis access or other incidents.

The timeline 410 can visually represent the patient's dialysis history over a set period of time. For example, the timeline 410 of FIG. 4 can show the previous twelve months. Various rows can represent different events during that time period. For example, the top row can represent balloons used during dialysis treatment. The middle row can represent use of a stent with the patient. The bottom row can show de-clotting procedures. Graphically, these incidents can be represented by icons, such as balloons, or by symbols of different types. In some cases, the timeline 410 can be linked to an overlay, such as the overlay 300A or 300B in FIGS. 3A and 3B. In this case, clicking on various points on the timeline 410 can automatically update the overlay to display the condition of the patient's access points at a particular time. In some cases, the timeline 410 can be used to dynamically update the overlay through sliding, tapping, or other selection methods. The dials 420, 440, can similarly update with a change in selection on the timeline 410. In this case, the relevant date for that data can be displayed.

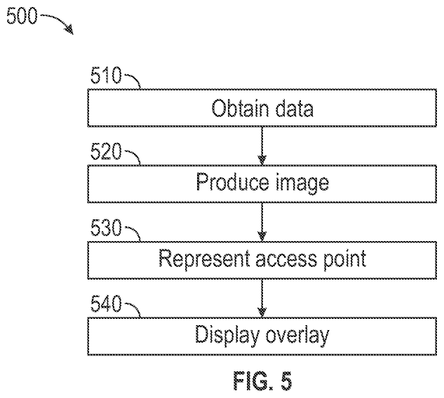

FIG. 5 illustrates a flow chart of a method 500 of administering dialysis in an example. Method 500 can include operations 510 to 540. The method 500 can including obtaining data (operation 510), producing an image (operation 520), representing access points (operation 530), and displaying the overlay (operation 540).

At operation 510, the overlay application can obtain data regarding access points. For example, the application can receive or retrieve data, such as from a database, indicating at least one dialysis access point on the patient. The data can include coordinates indicating a location relative to coordinates of an image, and a time stamp or date of when the access point was used.

At operation 520, the overlay application can produce an image of the location on the patient that has been used for dialysis access. For example, the overlay application can retrieve an image of a patient arm, such as from an outside application or database. In some cases, a leg, limb, or other location of the patient's body used for administration of dialysis can be depicted. The image can be, for example, a photographic image, or a computer-generated image. The image can be captured, for example, by a camera or other image capture device; or alternatively be partially or fully computer generated.

At operation 530, the overlay application can graphically represent the access point data. For example, the application can produce various markers at coordinates representative of each of the access points. In some cases, the markers can be a variety of symbols, such as representing different treatment times or dates. In some cases, the markers can be a variety of colors representing different treatment times or dates.

At operation 540, the overlay application can display the overlay, including the image and the markers, for reference by a user. The overlay can be displayed, for example, on a computer, tablet, or other appropriate device.

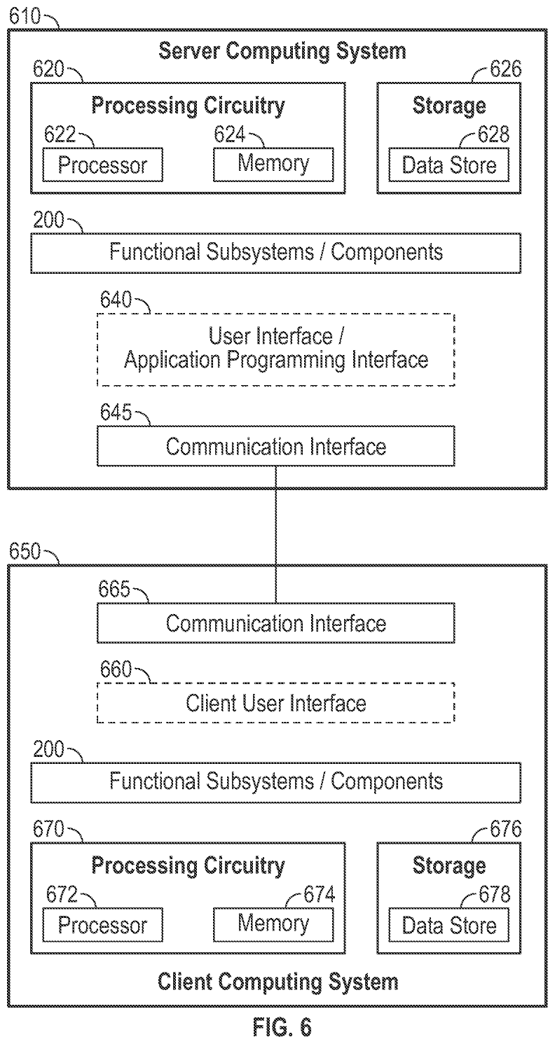

FIG. 6 illustrates a block diagram of an example server data processing system 610 and an example client computing system 650 for implementing the computing and processing techniques discussed herein, according to an example. The computing systems 610, 650 may be operated by or embodied in a number of different computing platforms, such as in a server form factor, a workstation, console, or personal computer form factor, a mobile computing device, etc., but are specifically illustrated with reference to server and client implementations. In some examples, the server system 610 may be a networked device connected via a network (or combination of networks) to the client computing system 650, with the server computing system operating an interface 640 (in the form of a server-hosted user interface, or an application programming interface) that is communicatively coupled to the network with a communication interface 645, and with the client computing system 650 operating a client user interface 660 that is communicatively coupled to the network with a communication interface 665. The network may include local, short-range, or long-range networks, such as Bluetooth, cellular, IEEE 802.11 (Wi-Fi), or other wired or wireless networks. For instance, a third party (e.g., dialysis service provider) may host and operate the server system 610, for access by the client computing system 650 operated by a dialysis provider at each dialysis point of treatment.

The systems 610, 650 each include a processor 622, 672 and a memory 624, 674, which can be arranged as part of processing circuitry 620, 670. The processors 622, 672 respectively may be any single processor or group of processors that act cooperatively. The memory 624, 674 respectively may be any type of memory, including volatile or non-volatile memory. The memory 624, 674 on each respective system may include instructions (e.g., provided by storage 626, 676), which when executed by the processor 622, 672 cause the processor 622, 672 to implement the functional features described herein. This may include, for the system 100 with overlay application 110, and database 120, and/or the system 200 with data sources or components 205, 210, 215, 220, 225, 230, 235, 240, and 245.

The systems may also include the generation, access, use, or persisting of data with use of data stores (e.g., databases) 628, 678. Thus, it will be understood that the references to electronic operations in the systems 610, 650 or the processing circuitry 620, 670 or software discussed herein may be performed by the respective processors 622, 672 or the circuitry 620, 670 as a whole. For example, the processors or circuitry may implement any of the features of the method 500 or functions described among the figures above, including those for operation of the user interfaces, application programming interfaces, and software or functional components. The processors or circuitry may further provide (or obtain) data and commands to assist the processing and implementation using communication interfaces 645, 665.

In various examples, the functional or data processing components discussed herein, such as those depicted in FIG. 2, can be implemented in different (or the same) computing systems (e.g., a single server, a collection of servers, a cloud-based computing platform, etc.). A computing system may comprise one or more processors that execute software instructions, such as those used to define a software or computer program, stored in a computer-readable storage medium such as a memory device (e.g., a main memory and a static memory, a Flash memory, random access memory (RAM), or any other type of volatile or non-volatile memory that stores instructions), or a storage device (e.g., a disk drive, or an optical drive). Alternatively, or additionally, the computing system may comprise dedicated hardware, such as one or more integrated circuits, one or more Application Specific Integrated Circuits (ASICs), one or more Field Programmable Gate Arrays (FPGAs), artificial intelligence (AI) or machine learning (ML) acceleration circuitry, or any combination of the foregoing examples of dedicated hardware, for performing the techniques described in this disclosure.

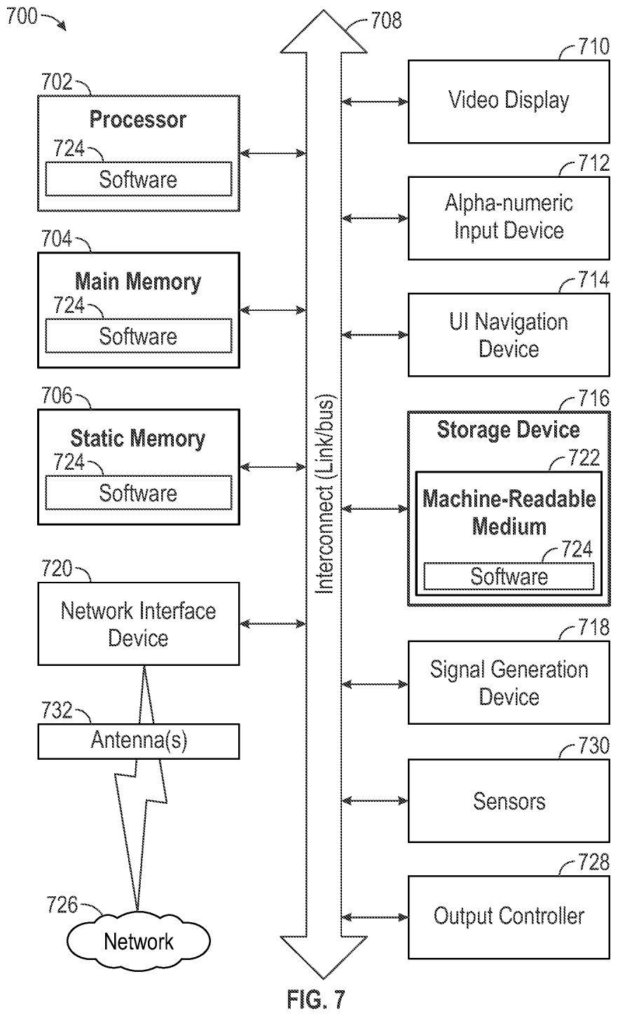

FIG. 7 illustrates a block diagram of an example computing system machine 700 upon which any one or more of the techniques (e.g., methodologies) discussed herein may perform. Machine 700 (e.g., computer system) may include a hardware processor 702 (e.g., a central processing unit (CPU), a graphics processing unit (GPU), a hardware processor core, or any combination thereof), a main memory 704 and a static memory 706, connected via an interconnect 708 (e.g., link or bus), as some or all of these components may constitute hardware for systems 100 or 200 or hardware to operate the services and subsystems and related implementations discussed above.

Specific examples of main memory 704 include Random Access Memory (RAM), and semiconductor memory devices, which may include, in some embodiments, storage locations in semiconductors such as registers. Specific examples of static memory 706 include non-volatile memory, such as semiconductor memory devices (e.g., Electrically Programmable Read-Only Memory (EPROM), Electrically Erasable Programmable Read-Only Memory (EEPROM)) and flash memory devices: magnetic disks, such as internal hard disks and removable disks: magneto-optical disks: RAM; and CD-ROM and DVD-ROM disks.

The machine 700 may further include a display device 710, an input device 712 (e.g., a keyboard), and a user interface (UI) navigation device 714 (e.g., a mouse). In an example, the display device 710, input device 712 and UI navigation device 714 may be a touch screen display. The machine 700 may additionally include a mass storage device 716 (e.g., drive unit), a signal generation device 718 (e.g., a speaker), a network interface device 720, and one or more sensors 730, such as a global positioning system (GPS) sensor, compass, accelerometer, or some other sensor. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.). In some embodiments the hardware processor 702 and/or instructions 724 may comprise processing circuitry and/or transceiver circuitry.

The mass storage device 716 may include a machine readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the mass storage device 716 constitutes, in at least some embodiments, machine readable media.

The term “machine readable medium” includes, in some embodiments, any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Specific examples of machine-readable media include, one or more of non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks: magneto-optical disks: RAM; and CD-ROM and DVD-ROM disks. While the machine readable medium 722 is illustrated as a single medium, the term “machine readable medium” includes, in at least some embodiments, a single medium or multiple media (e.g., a centralized or distributed database, and/or associated caches and servers) configured to store the one or more instructions 724. In some examples, machine readable media includes non-transitory machine-readable media. In some examples, machine readable media includes machine readable media that is not a transitory propagating signal.

The instructions 724 are further transmitted or received, in at least some embodiments, over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®), IEEE 802.15.4 family of standards, a Long Term Evolution (LTE) 4G or 5G family of standards, a Universal Mobile Telecommunications System (UMTS) family of standards, peer-to-peer (P2P) networks, satellite communication networks, among others.

An apparatus of the machine 700 includes, in at least some embodiments, one or more of a hardware processor 702 (e.g., a central processing unit (CPU), a graphics processing unit (GPU), a hardware processor core, or any combination thereof), a main memory 704 and a static memory 706, sensors 730, network interface device 720, antennas 732, a display device 710, an input device 712, a UI navigation device 714, a mass storage device 716, instructions 724, a signal generation device 718, and an output controller 728. The apparatus is configured, in at least some embodiments, to perform one or more of the methods and/or operations disclosed herein. The apparatus is, in some examples, a component of the machine 700 to perform one or more of the methods and/or operations disclosed herein, and/or to perform a portion of one or more of the methods and/or operations disclosed herein.

In an example embodiment, the network interface device 720 includes one or more physical jacks (e.g., Ethernet, coaxial, or phone jacks) or one or more antennas to connect to the communications network 726. In an example embodiment, the network interface device 720 includes one or more antennas 732 to wirelessly communicate using at least one of single-input multiple-output (SIMO), multiple-input multiple-output (MIMO), or multiple-input single-output (MISO) techniques. In some examples, the network interface device 720 wirelessly communicates using Multiple User MIMO techniques. The term “transmission medium” shall be taken to include any intangible medium that is capable of storing, encoding or carrying instructions for execution by the machine 700, and includes digital or analog communications signals or other intangible medium to facilitate communication of such software.

At least some example embodiments, as described herein, include, or operate on, logic or a number of components, modules, or mechanisms. Such components are tangible entities (e.g., hardware) capable of performing specified operations and are configured or arranged in a certain manner. In an example, circuits are arranged (e.g., internally or with respect to external entities such as other circuits) in a specified manner as a module. In an example, the whole or part of one or more computer systems (e.g., a standalone, client or server computer system) or one or more hardware processors are configured by firmware or software (e.g., instructions, an application portion, or an application) as a component that operates to perform specified operations. In an example, the software resides on a machine readable medium. In an example, the software, when executed by the underlying hardware of the component, causes the hardware to perform the specified operations.

Accordingly, such components are understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which components are temporarily configured, each of the modules need not be instantiated at any one moment in time. For example, where the components comprise a general-purpose hardware processor configured using software, in some embodiments, the general-purpose hardware processor is configured as respective different components at different times. Software accordingly configures a hardware processor, for example, to constitute a particular component at one instance of time and to constitute a different component at a different instance of time.

Some embodiments are implemented fully or partially in software and/or firmware. This software and/or firmware takes the form of instructions contained in or on a non-transitory computer-readable storage medium, in at least some embodiments. Those instructions are then read and executed by one or more hardware processors to enable performance of the operations described herein, in at least some embodiments. The instructions are in any suitable form, such as but not limited to source code, compiled code, interpreted code, executable code, static code, dynamic code, and the like. Such a computer-readable medium includes any tangible non-transitory medium for storing information in a form readable by one or more computers, such as but not limited to read only memory (ROM): random access memory (RAM): magnetic disk storage media: optical storage media: flash memory, etc.

Various embodiments may be implemented fully or partially in software and/or firmware. This software and/or firmware may take the form of instructions contained in or on a non-transitory computer-readable storage medium. Those instructions are then read and executed by one or more processors to enable performance of the operations described herein. The instructions are in any suitable form, such as but not limited to source code, compiled code, interpreted code, executable code, static code, dynamic code, and the like. Such a computer-readable medium includes, in at least some embodiments, any tangible non-transitory medium for storing information in a form readable by one or more computers, such as but not limited to read only memory (ROM): random access memory (RAM): magnetic disk storage media; optical storage media: flash memory, etc.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-25. (canceled)

26. A computer-implemented method of producing an overlay of dialysis access locations, the method comprising:
obtaining data indicating at least one dialysis access component and at least one dialysis access point of a patient, each of the at least one dialysis access component having an associated location on the patient and an associated type, and each of the at least one dialysis access point having an associated location on the patient and an associated access time;
obtaining an image of an anatomical area of the patient that is used for dialysis administration;
graphically representing the data as an overlay on the image of the anatomical area, the overlay depicting:
each of the at least one dialysis access component as an icon on the image of the anatomical area at a corresponding component, wherein the icon conveys both location and type of the corresponding component; and
each of the at least one dialysis access point as a marker on the image of the anatomical area at a corresponding access point, wherein the marker conveys both location and time of use of the corresponding access point; and
displaying the image of the anatomical area to a user via a user interface, including the overlay that depicts each of the at least one dialysis access component and each of the at least one dialysis access point.

27. The method of claim 26, wherein the at least one dialysis access component corresponds to at least one graft, stent, or arteriovenous fistula.

28. The method of claim 26, further comprising graphically representing at least one medical incident on the image, wherein the at least one medical incident is associated with a balloon dilation, pseudoaneurysm, aneurysm, rupture, stenosis, arterial anastomosis, or venous anastomosis.

29. The method of claim 26, wherein the marker corresponds to a keep-out area within the anatomical area to avoid for new administration of dialysis, wherein at least one of size, color, or shape of the marker are based on characteristics of the keep-out area.

30. The method of claim 26, wherein the marker comprises a symbol or color associated with the access time of each corresponding dialysis access point.

31. The method of claim 30, wherein the symbol or color changes over time to convey information about an elapsed time since dialysis administration with each corresponding dialysis access point.

32. The method of claim 30, wherein graphically representing the data includes representing a first dialysis access point with a first marker and a second dialysis access point with a second marker, wherein the first marker comprises a first symbol or a first color associated with a first date of dialysis administration at the first dialysis access point, and wherein the second marker comprises a second symbol or a second color associated with a second date of dialysis administration at the second dialysis access point.

33. The method of claim 26, wherein each marker is positioned based on location coordinates of a corresponding access point within coordinates of the image of the anatomical area.

34. The method of claim 26, wherein obtaining the image comprises obtaining a photographic image of the anatomical area of the patient, the photographic image captured with a camera, producing a computer-generated image of the anatomical area of the patient, or both.

35. The method of claim 26, further comprising superimposing a radiographic layer on the image, the radiographic layer comprising another anatomical depiction of the anatomical area.

36. The method of claim 26, further comprising superimposing a text overlay on the image, the text overlay comprising annotations or notes associated with the at least one dialysis access point or the at least one dialysis access component.

37. The method of claim 26, further comprising recording a new dialysis access point after a new administration of dialysis, and updating the overlay by graphically representing the new dialysis access point as a new marker on the image of the anatomical area at a corresponding new dialysis access point, wherein the new marker conveys both location and date of the new dialysis access point.

38. The method of claim 26, further comprising graphically representing information in the overlay to indicate which dialysis access points were entered at which times.

39. The method of claim 26, further comprising recording dialysis administration information associated with particular locations along the at least one dialysis access component.

40. The method of claim 39, wherein the dialysis administration information is selectable as a visual display as an overlay of a photographic record of the patient when selected by a user, and wherein the dialysis administration information presents a distribution of physiologic parameters along a course of a graft or arteriovenous fistula corresponding to the at least one dialysis access component.

41. The method of claim 40, wherein the physiologic parameters include at least one of: a depth below skin to the at least one access component, dialysis flow rate through the at least one access component, dialysis flow rate obtained with needles positioned in various locations, or a rating or grading of ease of access at the at least one access point.

42. The method of claim 26, wherein obtaining data comprises receiving the data indicating the at least one dialysis access point from a database, and wherein the database stores data corresponding to performance metrics for prior access by each clinician at the at least one dialysis access point, the performance metrics being determined based on avoidance of prior dialysis access points by each clinician.

43. The method of claim 26, wherein the method is performed by a server computing system, and wherein displaying the overlay to the user comprises providing the image of the anatomical area from the server computing system to a client computing system operated by the user.

44. The method of claim 26, wherein the method is performed by a client computing system, and wherein displaying the image to the user comprises providing the image of the anatomical area in a graphical user interface provided by the client computing system, wherein the graphical user interface comprises a website interface, wherein the graphical user interface comprises a software application, and wherein the client computing system is a mobile computing device.

45. The method of claim 26, wherein the method is performed by an augmented reality device, wherein obtaining the image of the anatomical area comprises capturing the image of the anatomical area from a real-world view of the anatomical area, wherein displaying the overlay to the user comprises augmenting the real-world view of the anatomical area in a display provided by the augmented reality device, and wherein the augmented reality device is a headset, smart glasses, or a smartphone with integrated camera and display.

46. A machine-readable medium including instructions, which when executed by a processor of a computing system, cause the computing system to execute the method of claim 26.
